(19) United States
(12) Patent Application Publication
(10) Pub. No.: US 2024/0261648 A1
(43) Pub. Date: Aug. 8, 2024

(54) METHOD AND SYSTEM FOR PERSONALIZED CLUB FITTING UTILIZING SWING CAPTURE DATA

(71) Applicant: Worldwide Golf Cool Clubs LLC, Bethesda, MD (US)

(72) Inventor: Mark D. Timms, Scottsdale, AZ (US)

(73) Assignee: Worldwide Golf Cool Clubs LLC, Bethesda, MD (US)

(21) Appl. No.: 18/163,781

(22) Filed: Feb. 2, 2023

(51) Int. Cl. A63B 60/42; A63B 24/00; A63B 60/46; G06F 30/17

(52) U.S. Cl. CPC A63B 60/42; A63B 24/0006; A63B 60/46; G06F 30/17; A63B 2024/0012; A63B 2024/0068; A63B 2102/32; A63B 2220/833; A63B 2225/20; A63B 2225/50

(57) ABSTRACT

A system for personalized club fitting according to various embodiments of the present technology provides a golfer with an opportunity to obtain custom built golf clubs without having to visit a specialized location. The system includes a device for capturing unique swing data relating to a user's golf swing. The device may include a removable sensor configured to be connected to a generic golf club swung by the user. The captured swing data is then transferred to a processing system where a user swing profile is generated and compared against a set of performance data for golf club shaft and golf club heads. A set of shafts and club heads are selected that match the user swing profile. This data can then be provided directly to the user or uploaded to a club builder so that the clubs can be built for the user.

### BACKGROUND OF THE TECHNOLOGY

Personalized club fitting allows a player to have a custom set of golf clubs made that best matches the unique parameters of not only that player's height but also that player's swing profile. The fitting process generally requires a player to physically visit a fitting location where they may swing one or more clubs while a machine collects data on various parameters of their swing profile. Once data is collected, it can be analyzed, and personalized club and shaft recommendations can be made. The player may then select from one or more of the recommendations and have a custom set of golf clubs built.

A drawback of this process is that it requires the player to visit the fitting location. Fitting locations often employ high speed camera systems that can not only capture the player's swing from multiple angles, but also collect data relating to the golf ball itself. For example, the impact of the golf club on the ball imparts a spin rate to the golf ball and a sets the ball in motion with an angle relative to the ground (launch angle). The systems required to capture this data are sensitive and may be expensive to purchase and operate. As a result, fitting locations may be more common in larger metropolitan areas than in rural areas. This increases the difficulty for a player living outside of a city to access personalized golf clubs.

### SUMMARY OF THE TECHNOLOGY

A system for personalized club fitting according to various embodiments of the present technology provides a golfer with an opportunity to obtain custom built golf clubs without having to visit a specialized location. The system includes a device for capturing unique swing data relating to a user's golf swing. The device may include a removable sensor configured to be connected to a generic golf club swung by the user. The captured swing data is then transferred to a processing system where a user swing profile is generated and compared against a set of performance data for golf club shaft and golf club heads. A set of shafts and club heads are selected that match the user swing profile. This data can then be provided directly to the user or uploaded to a club builder so that the clubs can be built for the user.

### BRIEF DESCRIPTION OF THE DRAWINGS

A more complete understanding of the present technology may be derived by referring to the detailed description when considered in connection with the following illustrative figures. In the following figures, like reference numbers refer to similar elements and steps throughout the figures. For simplicity and clarity of illustration, elements in the figures are not necessarily drawn to scale.

FIG. 1 representatively illustrates a sensor connected to a golf club in accordance with an exemplary embodiment of the present technology;

FIGS. 2A-2E representatively illustrate portions of a swing in accordance with an exemplary embodiment of the present technology; and

FIG. 3 representatively illustrates a flow chart of the fitting process in accordance with an exemplary embodiment of the present technology.

### DETAILED DESCRIPTION OF THE DRAWINGS

The present technology may be described in terms of functional block components and various processing steps. Such functional blocks may be realized by any number of components configured to perform the specified functions and achieve the various results. For example, the present technology may employ various types of sensors, data capture devices, databases, measuring systems, and the like, which may carry out a variety of functions. Further, the present technology may employ any number of conventional techniques for measuring physical properties of one or more components of a golf club during use, comparing captured data to known data tables, predicting performance of an individual, and/or capturing and analyzing test data.

Methods and apparatus for personalized club fitting utilizing swing capture data according to various aspects of the present technology may operate in conjunction with any suitable sensor or data acquisition system. Various representative implementations of the present technology may be applied to any system for capturing elements of a particular's user's swing in real-time and providing the captured data to a remote system.

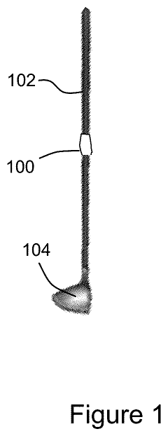

Referring now to FIG. 1, methods and apparatus for personalized club fitting utilizing swing capture data may comprise a sensor 100 attached to a golf club shaft 102. The sensor 100 may comprise and internal microprocessor and one or more internal data acquisition devices such as an accelerometer, inertial motion unit, gyro sensor, magnetometer that are configured to capture, record, or otherwise collect various elements and parameters of the user's swing such as acceleration levels throughout the entire swing, hand speed, rotation, timing intervals within stages of the swing, orientation of the club during the swing, clubhead speed at impact, or any other suitable parameter. The sensor 100 may also comprise a microcircuit having a controller, a battery, a communication device (e.g., a transceiver or radio), and a memory device.

The sensor 100 is configured to be selectively attachable and removable from a golf club at any suitable location. For example, in one embodiment the sensor may be configured to be fit over a butt end of the golf club opposite the clubhead 104. In an alternative embodiment, the sensor 100 may be removably connected to a mid-section of the club located between the butt end of club and the club head 104. In this way, the sensor 100 can be selectively connected to any desired golf club during a data collection process.

A microcircuit within the sensor 100 may be programmed to sense movement and position of the golf club in space and distinguish between general movement of the club and a swing that is representative of an actual golf swing taken during play. The microcircuit may also be programmed to only collect swing data during a swing collection process. For example, an application interface may be communicatively linked to the sensor 100 and configured to generate a signal that is transmitted wirelessly to the sensor 100 to indicate that a data collection process is being initiated. In response, the microcircuit may activate one or more onboard data collection devices to begin monitoring and collecting swing data.

Collected data may be stored on the memory device of the sensor 100 or instantly transmitted to a remote storage device linked to the application interface or remote storage system. For example, in one embodiment, the application interface may be installed on a user's desktop, portable computing device, or mobile phone. Collected data may be transmitted from the sensor 100 to the user's computing device for storage over any suitable wireless network such as WiFi or Bluetooth®. Alternatively, the application interface may be configured to upload the collected data to a remote storage device such as a cloud server.

A data processing system may be communicatively linked to the application interface and/or the sensor 100 itself and be configured to receive stored swing data over a communication network for processing. Alternatively, the sensor 100 may comprise a data port that may allow for a direct data connection between the sensor 100 and the data processing system. The data processing system may comprise any suitable system or components for receiving transmitted data, processing the collected data, and generating a personalized custom club recommendation for the user.

The data processing system may comprise or be communicatively linked to one or more databases that contain calculated metrics for golf club components such as golf club shafts and club heads. The data processing system may process the received swing data and then perform a comparative search of the database to determine a set of fitting data that comprises an appropriate combination of components best suited for the user according to their unique swing.

The determined set of fitting data may then be used to custom build one or more golf clubs for the user. For example, the user may be provided with the set of fitting data that they could take to a custom club builder of their choice or the set of fitting data may be transmitted from the data processing system directly to a club builder, original equipment manufacturer, or other third party so that it can be used to build a customized golf club or set of golf clubs.

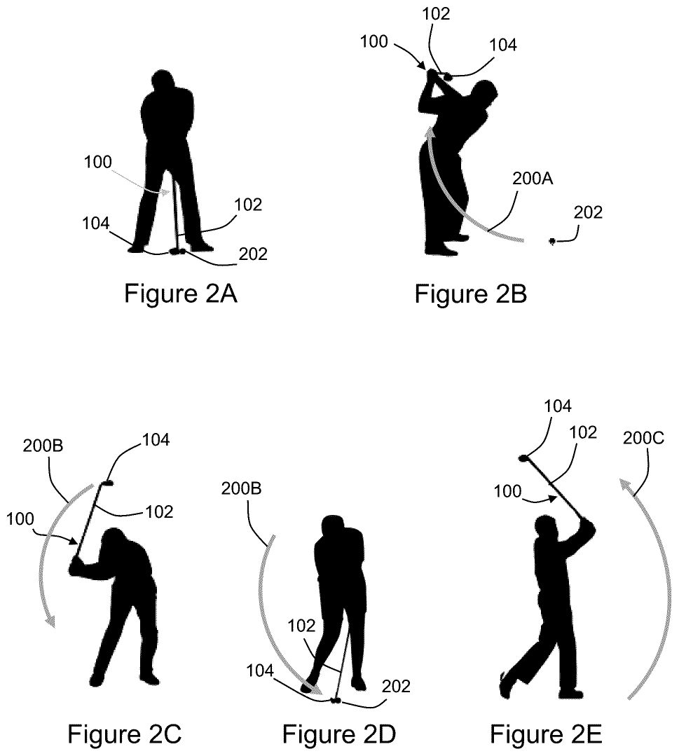

Referring now to FIGS. 2A-2E, the during a swing data capture session, the sensor 100 may be connected to the golf club shaft 102. With particular reference to FIG. 2A, the sensor 100 may be configured to identify when a user is addressing a golf ball 202 and preparing to hit it. This situation may be determined according to the orientation in space of the sensor 100, shaft 102, and club head 104. More specifically, the sensor 100 may be able to determine the relative position, angle, and motion of the shaft 102 and the club head 104 in space at any given time during the swing data capture session and be able to identify when the combination of elements matches a condition predetermined to be the address position prior to swinging the golf club.

Alternatively, the sensor 100 may be responsive to a video feed. For example, a user may be prompted by the application interface to position their mobile phone such that it is able to record a video of the user while they are swinging. The sensor 100 may be configured to detect if the video feed shows the user addressing the ball (as shown in FIG. 2A) and initiate a “ready state” for capturing swing data.

In yet another embodiment, the sensor 100 may be configured to record all data from the internal data acquisition devices once a swing data capture session is initiated and store the data in a buffer of memory. The sensor may further be configured to determine when an actual swing has taken place and then once the swing is completed access the buffer of memory to recall collected data from a period of time corresponding to the swing and save that recalled data as swing data.

To enable the sensor 100 to properly collect swing data, the user may be required to enter details about the particular golf club being swung. For example, before beginning the swing data capture session the user may be prompted to enter what club the user is swinging (e.g., driver, 3 wood, 5 iron, etc.), whether the golf club is right handed or left handed, and the length of the shaft 102. This data may be used to better calibrate the sensor 100 or help identify any differences in the user's swing from club-to-club.

Referring now to FIGS. 2A and 2B, at address, the sensor 100 may begin collecting swing data as the backswing 200A starts. The sensor 100 may collect any suitable data such as speed of the shaft 102 and/or club head 104, the user's hand speed, any rotation of the shaft 102 and club head 104 as the backswing 200A progresses, acceleration, a swing path, a swing plane, or any other suitable data as a function of time.

Referring now to FIGS. 2C and 2D, after the backswing 200A is completed the user begins a downswing 200B wherein the user is now moving the shaft 102 and club head 104 in an arcing motion downwardly to strike the golf ball 202. During the downswing 200B the sensor 100 may continue collecting the same data as during the backswing 200A. The sensor 100 may also collect and additional data elements such as flex of the shaft 102, variations from measurements of the backswing 200A, or any other suitable criteria.

With particular reference to FIG. 2D, at impact, the sensor 100 may detect the moment when the club head 104 strikes the golf ball 202. This detected moment may be noted by the sensor 100, the application interface, or the data processing system such that certain elements of the swing, such as speed at impact, angle of the shaft 102 at impact, angle of attack, club head path pre and post impact, acceleration, or any other elements can be noted for analysis by the data processing system and/or presentation to the user for evaluation.

Now referring to FIG. 2E, the sensor 100 may continue collecting data after impact during the follow thru 200C portion of the swing. During this period the swing path/plane is completed and the shaft 102 and club head 104 decelerate. Once the follow thru 200C is completed, the sensor may cease collecting swing data for that swing or otherwise be configured to identify the end of the swing from a stream of continuously logged data. The collected data may then be stored with additional swing data or sent directly to the application interface, data processing system, or cloud-based server for storage or processing.

The data processing system may process the swing data of any number of swings for a given user to generate a user swing profile. A general swing profile may be generated, or the swing profile may include multiple swing profiles that are specific to a particular type of golf club (e.g., wood vs. iron). The user swing profile may then be processed against one or more databases to identify one or more shafts 102 and/or club heads 104 that best match the user's swing profile. Additional elements such as user preferences or style of play may also be included in the analysis.

The user's swing profile may comprise one or more calculated elements based on the swing data such as a value of range of shaft 102 stiffness or shaft flex that best matches the user's swing. For example, as the downswing 200B progresses some parameters such as speed and acceleration may create additional forces on the shaft 102 causing it to flex (bend) prior to impacting the golf ball 202. The amount of flex in the club shaft 102 is directly impacted by factors such as speed, acceleration, and the construction of the golf shaft 102 itself. Specific flex and/or stiffness values may be stored in the database and may be provided by the manufacturer or determined according to actual testing data. For example, the data processing system may be linked to one or more databases that includes actual test data for individual golf shafts 102 and club heads 104.

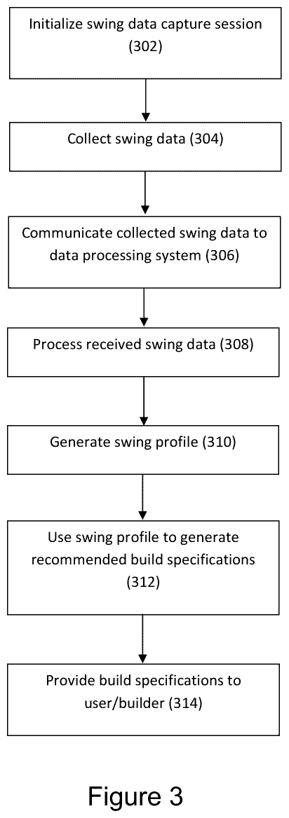

In use, and with reference now to FIG. 3, the system may allow a user to begin a swing data capture session (302) in which the user swings a golf club with an attached sensor 100. The sensor 100 is configured to capture swing data for any given number of swings by the user (304). Collected swing data may then be communicated to a data processing system either directly or through an application interface communicatively linked to the sensor 100 (306).

After the data processing system receives the swing data, it may process the data (308) to generate a swing profile for the user (310). The data processing system may then compare the generated swing profile against one or more databases to determine a recommended set of build specifications for one or more golf clubs that are unique to that user (312). The data processing system may be further configured to determine any additional adjustments that a given club may need based on the user's swing profile. For example, the data processing system may determine an optimized swing weight for each club in the set of build specifications. The build specifications may then be communicated to the user and/or a club builder (314) so that the user can have one or more golf clubs built that are optimized to their swing according to the collected data.

For example, one database accessed by the data processing system may comprise performance characteristics of individual golf shafts from one or more original equipment manufacturers. The performance characteristics included in the database may include manufacturers specifications, results of robotic testing, laboratory testing, and real-world test results relating to parameters such as: a stiffness profile for each shaft, a torque profile for each shaft, a weight of the shaft, a straightness score for each shaft, and any other suitable criteria for evaluating the performance of a golf shaft. The data processing system may be configured to identify one or more golf club shafts that are a good match for the user's swing profile and include that into the set of build specifications.

Another representative database that may be used by the data processing system may include the performance characteristics of individual golf clubheads from one or more original equipment manufacturers. Additional performance characteristics may be obtained from results of robotic testing, laboratory testing, and real-world test results. Individual parameters included in this database may include any suitable criteria for matching a given club head to a user's swing profile such as: the type of club head (e.g., blade or cavity back); size (volume) of the club head, the loft of the club head, the lie of the club head.

Feedback from professional club fitters may also be incorporated into one or more of the databases to further refine the data available to the data processing system for generating customized build specifications. The data processing system may be configured to identify one or more golf club heads that are a good match for the user's swing profile and include that into the set of build specifications.

Due to the removability of the sensor 100 from a first golf club and connection to and use on another golf club, the system may be used by multiple users to generate unique swing profiles. The swing capture session may be conducted in a standard testing cell common at many retail golf locations. These retail locations may be able to provide a better fitting club to the user by taking into account swing data elements that are not collected through current methods.

The disclosed technology also provides a unique application that is not found in current club fitting systems. More specifically, the technology allows a user to capture swing data away from a retail location and without the need to use expensive high speed video cameras. For example, in one embodiment, a user may be able to request a sensor 100 from a club builder, have the sensor 100 mailed to them, and conduct a swing capture session at the time and place of the user's choosing. For example, the user may connect the sensor 100 to their golf club and take swings in their own backyard, a driving range, or even during an actual round of golf. After the data has been collected, the sensor 100 may upload the data to an application interface on the user's smartphone so that the data can be transferred to a remote server or the user may send the sensor 100 back to the club builder who can then download the collected swing data from the sensor 100 to an application interface or upload the collected swing data from the sensor 100 directly to the data processing system. The club builder may then get the results and build the user a set of golf clubs. In an alternate embodiment, the user may request a sensor 100 and use it as described above, but then receive the results of the analysis. These results may then be taken by the user to the club builder of their choice. The disclosed system opens up the ability to obtain customized golf clubs for users who do not live near a custom club builder or do not want to visit a retail location.

The application interface may also be configured to receive data from other sources than the sensor 100. For example, the sensor 100 may be configured to capture video data acquired by the user's smart phone or other computing device during a swing capture session. This acquired data may be used to create the swing profile in place of or in addition to data captured by the sensor 100. In one embodiment, the application interface may be configured to calculate factors such as clubhead speed at impact, attack angle, and an approximate launch angle based on video data alone. This information may be used to create a swing profile absent additional sensor 100 data, though the result may not be as accurate. This may allow for varying levels of club fitting where, for example, a basic customized club may be built based on data collected by the application interface alone and a more advanced customized club may be built that utilizes data from the sensor 100.

The particular implementations shown and described are illustrative of the technology and its best mode and are not intended to otherwise limit the scope of the present technology in any way. Indeed, for the sake of brevity, conventional manufacturing, connection, preparation, and other functional aspects of the system may not be described in detail. Furthermore, the connecting lines shown in the various figures are intended to represent exemplary functional relationships and/or steps between the various elements. Many alternative or additional functional relationships or physical connections may be present in a practical system.

In the foregoing description, the technology has been described with reference to specific exemplary embodiments. Various modifications and changes may be made, however, without departing from the scope of the present technology as set forth. The description and figures are to be regarded in an illustrative manner, rather than a restrictive one and all such modifications are intended to be included within the scope of the present technology. Accordingly, the scope of the technology should be determined by the generic embodiments described and their legal equivalents rather than by merely the specific examples described above. For example, the steps recited in any method or process embodiment may be executed in any appropriate order and are not limited to the explicit order presented in the specific examples. Additionally, the components and/or elements recited in any system embodiment may be combined in a variety of permutations to produce substantially the same result as the present technology and are accordingly not limited to the specific configuration recited in the specific examples.

Benefits, other advantages and solutions to problems have been described above with regard to particular embodiments. Any benefit, advantage, solution to problems or any element that may cause any particular benefit, advantage or solution to occur or to become more pronounced, however, is not to be construed as a critical, required or essential feature or component.

As used herein, the terms “comprises,” “comprising,” or any variation thereof, are intended to reference a non-exclusive inclusion, such that a process, method, article, composition or apparatus that comprises a list of elements does not include only those elements recited but may also include other elements not expressly listed or inherent to such process, method, article, composition or apparatus. Other combinations and/or modifications of the above-described structures, arrangements, applications, proportions, elements, materials or components used in the practice of the present technology, in addition to those not specifically recited, may be varied or otherwise particularly adapted to specific environments, manufacturing specifications, design parameters or other operating requirements without departing from the general principles of the same. Any terms of degree such as “substantially,” “about,” and “approximate” as used herein mean a reasonable amount of deviation of the modified term such that the end result is not significantly changed. For example, these terms can be construed as including a deviation of at least ±5% of the modified term if this deviation would not negate the meaning of the word it modifies.

The present technology has been described above with reference to an exemplary embodiment. However, changes and modifications may be made to the exemplary embodiment without departing from the scope of the present technology. These and other changes or modifications are intended to be included within the scope of the present technology.

1. A method for obtaining a set of instructions for building a customized golf club based on a user swinging a generic golf club, comprising:
providing a removable sensor to the user;
attaching the removable sensor to a shaft of the generic golf club;
establishing a communication link between the sensor and an application interface;
sensing movement data and position data of the generic golf club with the sensor to differentiate between general movement of the generic golf club and swinging of the generic golf club that is representative of an actual golf swing;
when the sensor senses the swinging of the generic golf club, initiating a swing data capture session;
capturing a set of swing data with the sensor in response to the user swinging the generic golf club;
transferring the captured swing data to a data processing system, wherein the data processing system is configured to:
analyze the transferred swing data to determine a user swing profile;
compare the user swing profile to a set of shaft data corresponding a set of golf shafts; and
select at least one golf shaft from the set of golf shafts with performance characteristics matching the user swing profile; and
communicate the selected golf shaft.

2. A method for obtaining a set of instructions for building a customized golf club according to claim 1, wherein the data processing system is further configured to:
compare the user swing profile to a set of head data corresponding a set of golf club heads; and
select at least one golf club head from the set of golf club heads with performance characteristics matching the user swing profile.

3. A method for obtaining a set of instructions for building a customized golf club according to claim 1, wherein the performance characteristics for the set of shaft data comprises:
a stiffness profile for each shaft;
a straightness score for each shaft; and
a torque profile for each shaft.

4. A method for obtaining a set of instructions for building a customized golf club according to claim 1, wherein the application interface is configured to:
be loaded onto a user device;
receive the captured swing data from the sensor; and
transmit the swing data over a network to the data processing system.

5. A method for obtaining a set of instructions for building a customized golf club according to claim 1, wherein:
the captured swing data is saved on the sensor; and
the sensor is communicatively linked to the data processing system prior to transferring the captured swing data to a data processing system.

6. A method for obtaining a set of instructions for building a customized golf club according to claim 5, wherein the application interface is configured to link the sensor to the data processing system and facilitate the transfer of the captured swing data.

7. A method for obtaining a set of instructions for building a customized golf club according to claim 1, wherein the sensor comprises:
a microprocessor;
a controller;
an internal battery;
a communication device configured to communicate with the application interface;
a memory device; and
an internal data acquisition device comprising at least of an accelerometer, inertial motion unit, gyro sensor, and magnetometer.

8. A system for obtaining a set of instructions for building a customized golf club based on a user swinging a generic golf club, comprising:
a sensor configured to be temporarily connected to the generic golf club, wherein the sensor is configured to:
sense movement data and position data of the generic golf club to differentiate between general movement of the generic golf club and swinging of the generic golf club that is representative of an actual golf swing;
initiate a swing data capture session in response to sensing the swinging of the generic golf club; and
capture a set of swing data in response to the user swinging the generic golf club;
an application interface configured to be communicatively linked to the sensor, wherein the application interface provides the signal to initiate the swing data capture session; and
a data processing system configured to:
receive the captured swing data;
analyze the transferred swing data to determine a user swing profile;
compare the user swing profile to a set of shaft data corresponding a set of golf shafts; and
select at least one golf shaft from the set of golf shafts with performance characteristics matching the user swing profile.

9. A system for obtaining a set of instructions for building a customized golf club according to claim 8, wherein the data processing system is further configured to:
compare the user swing profile to a set of head data corresponding a set of golf club heads; and
select at least one golf club head from the set of golf club heads with performance characteristics matching the user swing profile.

10. A system for obtaining a set of instructions for building a customized golf club according to claim 8, wherein the performance characteristics for the set of shaft data comprises:
a stiffness profile for each shaft;
a straightness score for each shaft; and
a torque profile for each shaft.

11. A system for obtaining a set of instructions for building a customized golf club according to claim 8, wherein the application interface is configured to:
be loaded onto a user device;
receive the captured swing data from the sensor; and
transmit the swing data over a network to the data processing system.

12. A system for obtaining a set of instructions for building a customized golf club according to claim 8, wherein:
the captured swing data is saved on the sensor; and
the sensor is communicatively linked to the data processing system prior to transferring the captured swing data to a data processing system.

13. A system for obtaining a set of instructions for building a customized golf club according to claim 12, wherein the application interface is configured to link the sensor to the data processing system and facilitate the transfer of the captured swing data.

14. A system for obtaining a set of instructions for building a customized golf club according to claim 8, wherein the sensor comprises:
a microprocessor;
a controller;
an internal battery;
a communication device configured to communicate with the application interface;
a memory device; and
an internal data acquisition device comprising at least of an accelerometer, inertial motion unit, gyro sensor, and magnetometer.
